(19) United States
(12) Patent Application Publication
(10) Pub. No.: US 2013/0143276 A1
(43) Pub. Date: Jun. 6, 2013

(54) Compositions and Methods for Adenylating Oligonucleotides

(71) Applicants: Alexander Zhelkovsky, Peabody, MA (US); Larry A. McReynolds, Beverly, MA (US)

(72) Inventors: Alexander Zhelkovsky, Peabody, MA (US); Larry A. McReynolds, Beverly, MA (US)

(73) Assignee: NEW ENGLAND BIOLABS, INC., Ipswich, MA (US)

(21) Appl. No.: 13/638,088

(22) PCT Filed: Apr. 1, 2011
(86) PCT No.: PCT/US11/30881

§ 371 Date: Oct. 24, 2012

Related U.S. Application Data
(60) Provisional application No. 61/320,203, filed on Apr. 1, 2010.
(60) Provisional application No. 61/427,179, filed on Dec. 25, 2010.
(60) Provisional application No. 61/427,178, filed on Dec. 25, 2010.

(51) Int. Cl. C12P 19/34

(52) U.S. Cl. CPC C12P 19/34
USPC 435/91.52

(57) ABSTRACT

A method is provided for generating a preparation in which more than 70% of the oligonucleotides are adenylated. The method includes reacting an oligonucleotide with an ATP-sensitive ligase where the ligase is characterized by its ability to efficiently generate adenylated oligonucleotides at ATP concentrations at which ligation and circularization of the oligonucleotide is minimal.

### BACKGROUND

The increasing demand for 5′-adenylated DNA linkers and adapters correlates with the rapid progress in high throughput Next Generation Sequencing (NGS) of small RNAs. The construction of cDNA libraries for NGS requires attachment of 5′ and 3′ sequencing platform specific oligonucleotide adapters for downstream amplification, attachment that is facilitated by use of 5′-adenylated oligonucleotide linkers and adapters.

For first strand cDNA synthesis, an oligonucleotide adapter is ligated to 3′-end of an RNA polynucleotide. Annealing an oligonucleotide complementary to this attached adapter provides a priming site for copying the target RNA by reverse transcriptase. This protocol requires dephosphorylation of the RNA prior to ligation to prevent self-circularization or concatamerization of the RNA substrate. Self-ligation of the 3′ oligonucleotide adapter is also blocked, in this case by modification at the 3′-terminus. When subsequent steps require ligation of an oligonucleotide to the 5′ end of the RNA polynucleotide, for example to provide a second priming site for amplification of the reverse transcriptase cDNA product, the RNA must be re-phosphorylated to allow adapter ligation.

Use of adenylated linkers under conditions in which adenosine monophosphate (AMP) is not transferred to nucleic acids removes the need to dephosphorylate RNA substrates prior to ligation and prevents unwanted ligation products. Pre-adenylated oligonucleotide 3′ adapters may be used as a substrate in a ligation reaction with no ATP and either T4 RNA Ligase 1 (T4 Rnl1) (Lau, et al. Science 294(5543): 858-862 (2001)) or truncated version of T4 RNA Ligase 2 (T4 Rnl2) (Hafner, et al. Methods 44(1): 3-12 (2008)). This approach requires the use of an adenylated oligonucleotide adapter (AppDNA) as the ligation donor. Current methods for synthesis of AppDNA include either chemical synthesis or enzymatic synthesis.

A commonly used chemical method for pre-adenylation involves coupling of adenosine 5′-phosphorimidazolidate to 5′-phosphorylated oligonucleotide in solution or during solid phase oligonucleotide synthesis (Pfeffer, et al., Curr Protoc Mol Biol Chapter 26, Unit 26.4, John Wiley & Sons, Inc.: Hoboken, N.J. (2005)); Dai, et al., Org Lett 11 (5): 1067-1070 (2009)). This chemical method does not result in quantitative conversion of phosphorylated substrate to adenylated product. Consequently, purification is required to separate two closely related DNAs differing by a single nucleotide, further reducing the yield of desired product, and increasing the overall time and expense required to produce adenylated oligonucleotides.

A commonly used enzymatic method relies on T4 DNA ligase, requiring a multi-step process to create adenylated single-stranded DNA linkers. Since this enzyme requires a double-stranded DNA substrate, the single-stranded DNA linker is first annealed to an appropriately fashioned complementary oligonucleotide, then treated with T4 DNA ligase in the presence of ATP to adenylate the linker, and finally purified from the complementary DNA (Chiuman et al, Bioorg Chem 30 (5): 332-349 (2002); Vigneault et al., Nat Methods 5 (9): 777-779 (2008); Patel et al., Bioorg Chem 36 (2): 46-56 (2008); and U.S. published application 2010/0062494). As with the chemical synthesis, the multi-step aspects of this process increase both the time and expense required for production of the adenylated product.

### SUMMARY

In an embodiment of the invention, a method is provided for generating an adenylated oligonucleotide preparation that includes providing oligonucleotides having a 5′ phosphate; reacting the oligonucleotide with an ATP-sensitive ligase in the presence of an effective amount of ATP; and obtaining a stable reaction product in which greater than 70% or 80% or 90% of the oligonucleotides are adenylated. The effective amount of ATP is sufficient to permit adenylation while at the same time inhibit circularization of single-stranded DNA. For example, the effective amount may be in the range of 5 μM-10 mM ATP.

In an embodiment of the invention, the ATP-sensitive ligase is an RNA ligase and is thermostable, such as Methanobacterium thermoautotrophicum RNA ligase (MthRnl).

Examples of ATP-sensitive ligases for use in embodiments of the method include ligases with at least 90% sequence similarity with one or more of the ligases obtained from: Methanobacterium thermoautotrophicum; Pyrococcus abyssii; phage KVP40; Deinococcus radiodurans; Autographica California; Rhodothermus marinus; and phage TS2126.

Other examples of ATP-sensitive ligases include ligases having at least 90% amino acid sequence similarity to SEQ ID NO:1 or SEQ ID NO: 2.

In another embodiment, the method may, in addition to generating adenylated oligonucleotides, include ligating these adenylated oligonucleotides to polynucleotides by means of a second ligase such as a T4 RNA ligase, or mutants thereof such as a truncated T4 RNA ligase.

In an embodiment of the invention, the oligonucleotide may have either a blocked 3′ end or a free hydroxyl group at the 3′ end. The adenylation may be performed at a temperature in the range of 37° C.-70° C. and at a temperature-adjusted pH range of 5.5-8.0 (at 25° C.).

### BRIEF DESCRIPTION OF THE DRAWINGS

FIGS. 1A-F show gels of a comparison of different RNA ligases for efficiency of oligonucleotide adenylation. The RNA ligases used were MthRnl, CircLigasetM (Epicentre Biotechnology, Madison, Wis.; U.S. Pat. No. 7,303,901, Blondel et al. Nucleic Acids Res. 33: 135-142 (2005)), T4 Rnl1 and T4 Rnl2 with two oligonucleotide substrates, pDNA17c-NH₂ (SEQ ID NO:7) (FIGS. 1A, C and F) and pDNA21-3bioTEG (SEQ ID NO:5) (FIGS. 1B, 1D and 1E). Single-stranded RNA size markers (Mr) were included for reference. Above each gel the molar ratio of substrate to enzyme (S/E) for each reaction is given. Molarity of MthRnl was calculated based on molecular weight of a monomer.

FIG. 1A shows adenylation of pDNA17c-NH₂ with MthRnl.

FIG. 1B shows adenylation of pDNA21-3bioTEG with MthRnl.

FIG. 1C shows adenylation of pDNA17c-NH₂ with CircLigase™.

FIG. 1D shows adenylation of pDNA21-3bioTEG with CircLigase™.

FIG. 1E shows adenylation of pDNA21-3bioTEG with T4 Rnl1 and T4 Rnl2.

FIG. 1F shows adenylation of pDNA17c-NH₂ with T4 Rnl1 and T4 Rnl2.

The gels in FIGS. 1A-1F show that all RNA ligases can adenylate an oligonucleotide to some extent although MthRnl and CircLigase™ were more effective than T4 Rnl1 and T4 Rnl2.

FIGS. 2A-2B show the dependence of DNA adenylation by MthRnl on ATP such that increasing concentrations of ATP produces greater adenylation of an oligonucleotide and reduced formation of circularized DNA. MthRnl (25 pmol of monomer) was reacted with 5 pmol of pDNA50 (SEQ ID NO:3) having an unprotected 3′-end for one hour using increasing concentrations of ATP (from left to right) as indicated below each lane.

FIG. 2A shows the inhibitory effect of ATP on ligation of pDNA50 at increasing concentrations. Circularization resulting from ligation was reduced with ATP concentrations of greater than 5 μM. When the concentration of ATP was increased to 50 μM, substantially complete adenylation of the oligonucleotide was achieved, and DNA circularization or ligation was almost completely inhibited.

FIG. 2B shows formation of an adenylated oligonucleotide (AppDNA17c-NH₂) in the presence of increasing amounts of ATP under otherwise constant conditions. Adenylation was complete at concentrations of greater than 10 μM ATP. In the 5′-adenylation of DNA with 3′ protected ends, AppDNA formation also increased with increasing ATP concentration and reached saturation near 50 μM.

FIG. 3 shows pH optimization of adenylation by MthRnl of an oligonucleotide in the presence of Mg ions. Oligonucleotide adenylation was measured at varying pHs. pDNA17c-NH₂ was adenylated by MthRnl most effectively in the pH range of 5.5-8.0. The size markers at the left of the gel (Mr) are single-stranded RNA. Optimum adenylation of the oligonucleotide occurred between pH 6.0 and 7.5.

FIGS. 4A-4G show that the oligonucleotide sequence and presence of a 3′-modification do not significantly influence the efficiency of adenylation of the substrate oligonucleotide. The MthRnl was 2-fold serially diluted as indicated by the substrate/enzyme ratio and reacted with 5 pmol of various oligonucleotides under standard reaction conditions for one hour. The molarity of the enzyme was calculated based on molecular weight of monomer of MthRnl. The substrates used in each of FIGS. 4A-4G are indicated.

FIG. 4H is a time-dependent assay using incubation periods of 0.5-4 hours and a single substrate to enzyme ratio corresponding to FIG. 4A, lane 3.

FIG. 5 shows a functional assay in which an adenylated oligonucleotide is ligated with an RNA acceptor using truncated T4 RNA Ligase 2 (T4 Rnl2tr also know as T4 RNA Ligase 2 [1-249]) without ATP under manufacturer's defined conditions. 10 μl ligation reactions containing 5 pmol of the RNA acceptor, 7 pmol AppDNA17c-NH₂ in 10 mM Tris-HCl pH 7.5 buffer, 10 mM Mg, 1 mM DTT and 200 U of T4Rnl2tr were incubated for 2 hours at 25° C. Reactions were stopped by adding 5 μl formamide loading buffer, heat-inactivated at 95° C. for 3 min, and products were separated, stained and visualized as described for DNA adenylation above. Mr is a marker lane. Lanes 1 and 3 contain non-ligated adenylated oligonucleotide and an RNA acceptor (RNA 22 ((SEQ ID NO:12)) or FAM-RNA 23 ((SEQ ID NO:11)), respectively) in the absence of T4Rnl2tr. Lanes 2 and 4 show ligation products of SEQ ID NO:12 or SEQ ID NO:11 ligated to AppDNA17c-NH₂ in the presence of T4Rnl2tr.

FIG. 6 shows a mass spectrometer analysis of the oligonucleotide pDNA21-NH₂ (SEQ ID NO:4) and its adenylated form after MthRnl treatment.

### DETAILED DESCRIPTION OF THE EMBODIMENTS

It was found that the use of certain ATP-dependent ligases, here referred to as ATP-sensitive ligases, is an efficient, low-cost solution for obtaining 5′-adenylated oligonucleotides. These ligases can be used in an improved high-yield method to generate adenylated oligonucleotides suitable as linkers, as compared with existing methods employing chemical synthesis or T4 DNA ligase. The high yield of adenylated oligonucleotide in the absence of ligation products obviates the need for gel purification to remove a template strand or incompletely modified substrates, thus reducing the cost of synthesis.

The term “ATP-sensitive ligase” as used herein refers to an ATP-dependent ligase, more particularly ATP-dependent RNA ligase, that can efficiently generate adenylated oligonucleotides at ATP concentrations where ligation and circularization of the oligonucleotide is minimal as determined by gel electrophoresis.

The term “oligonucleotide” as used herein refers to a single-stranded DNA.

The term “stable reaction product” as used herein refers to the ability of a ligase reaction product to be sufficiently stable as to be capable of visualization by gel electrophoresis after removal from a reaction vessel.

Embodiments of the method allow quantitative conversion of 5′-phosphorylated oligonucleotides to the adenylated form and do not require addition of a template strand for adenylation to occur. The high yields simplify isolation and purification of the adenylated product. The characteristics of ATP-sensitive ligases, under conditions of increased ATP concentrations, enable high efficiency adenylation of substrates (including substrates with 3′ unprotected ends) in the absence of ligation.

ATP-sensitive RNA ligases for use in the present embodiments may include: MthRnl (optimum 60-65° C.) (Torchia, et al. Nucleic Acids Res 36(19): 6218-6227 (2008)); Pyrococcus abyssii (PAB 1020) containing an archael (thermostable) RNA ligase; phage KVP40 RNA ligase (Yin et al. Virology 319: 141-151 (2004)); Deinococcus radiodurans RNA ligase (Raymond et al. Nucleic Acids Res 35: 839-849 (2007)); Autographica California RNA ligase (Martins et al. J. Biol. Chem. 279 (18): 18220-18231 (2004)); Rhodothermus marinus RNA ligase (Blondel et al. Nucleic Acids Res 31: 7247-7254 (2003)); and phage TS2126 containing CircLigase™. Other suitable ATP-sensitive ligases include recombinant enzymes derived from the above-cited host cells and mutants thereof. Related ATP-sensitive ligases may be identified by BLAST searches using the amino acid sequence of any known ATP-sensitive ligase or derivative thereof and used to adenylate oligonucleotides.

In an embodiment of the method, adenylated oligonucleotides may be produced in a reaction that includes an ATP-sensitive ligase and amounts of ATP of at least 5 μM ATP, for example at least 10 μM ATP, for example at least 20 μM ATP, for example at least 50 μM ATP, for example at least 75 μM ATP, for example at least 100 μM ATP and as much as 500 μM ATP, for example 750 μM, for example 1 mM ATP, for example 10 mM ATP.

In an embodiment of the method, a thermostable RNA ligase may be used such as an Mth RNA ligase for adenylating oligonucleotides at a temperature of reaction in the range of 37°-70° C., for example 55°-65° C.

In an embodiment of the method, the reaction buffer contains Mg⁺² or Mn⁺² as a cofactor.

In an embodiment of the method, a suitable pH for an ATP-sensitive ligase-mediated adenylation of an oligonucleotide varies according to the buffer and the temperature of the reaction. For example, in the presence of Mg ions, a pH may be used in the range of pH 5.5-8.0, for example pH 6.5-7.0; whereas in the presence of manganese, a pH may preferably be used in the range of 5.0-7.0, for example pH 5.5-6.0 (adjusted at 25° C.).

In an embodiment of the method, the reaction may be incubated for as long as 5 hours or more, or as short as 5 minutes, to obtain an adenylated oligonucleotide product. The incubation time appears to be approximately inversely correlated to the amount of the ATP-sensitive ligase in the reaction mixture.

The above-described ATP-sensitive ligases can be distinguished from a second group of ligases characterized by a T4 RNA ligase that does not efficiently adenylate oligonucleotides under conditions that disfavor ligation. Embodiments of the method illustrate how an ATP-sensitive ligase can be screened for its ability to adenylate an oligonucleotide where the adenylated product is suitable for ligation to another oligonucleotide using a ligase of the type exemplified by T4 RNA ligase. For example, T4 Rnl2tr is unable to transfer AMP to the 5′ phosphate of a nucleic acid, and thus is only capable of catalyzing ligation if the RNA or DNA is already adenylated. The use of adenylated oligonucleotides with T4 Rnl2tr allows selective ligation of DNA primers to RNA for cloning or sequencing (Ho, et al. Structure 12(2): 327-339 (2004); Nandakumar and Shuman, Molecular Cell 16(2): 211-21 (2004)).

With the goal of efficient synthesis of 5′-adenylated oligonucleotides, a simple one-step protocol using an ATP-sensitive ligase has been developed. Optimization of conditions for this one-step protocol can be determined by a person of ordinary skill in the art without undue experimentation using embodiments of the method and assays described in the examples for CircLigase™ and MthRnl. ATP-sensitive ligases from sources other than those described in the examples may be identified and their oligonucleotide adenylation activity optimized by substitution of the ATP-sensitive ligase in the screening assays described herein with the test ligase. Conditions for optimization may include pH, temperature, amount of ATP, ratio of substrate to enzyme, and salt type and concentration.

Under optimized conditions, the yield of adenylated oligonucleotides can be as much as 70%, 80%, 90%, 95% or 98% using ATP-sensitive ligases. The high yield eliminates the need for additional purification from unadenylated forms. However, if purification is desired, then the ATP-sensitive ligase can be removed, for example by heat-killing followed by a proteinase K digestion, extraction with phenol-chloroform-isoamyl alcohol and removal by HPLC, thus providing a purified linker population that contains 5′ App. The recovery of adenylated polynucleotide linkers may be as much as 70%, 80%, 90%, 95% or 98% of the starting amount of polynucleotide.

Some advantages of using a thermostable ATP-sensitive ligase include: (a) the enzyme can be purified in high yields from an overexpressing strain of E. coli; (b) oligonucleotides can be adenylated without the need for adding a complementary strand; and (c) secondary structures in the oligonucleotides are reduced at elevated temperatures, and have a lower potential to interfere with adenylation of the 5′ end of the oligonucleotide.

All references cited herein, as well as U.S. provisional application Ser. Nos. 61/320,203 filed Apr. 1, 2010, 61/427,179 filed Dec. 25, 2010 and 61/427,178 filed Dec. 25, 2010, are hereby incorporated by reference.

### EXAMPLES

### Example 1

### Screening Ligases

A simple one-step protocol for screening for ATP-sensitive

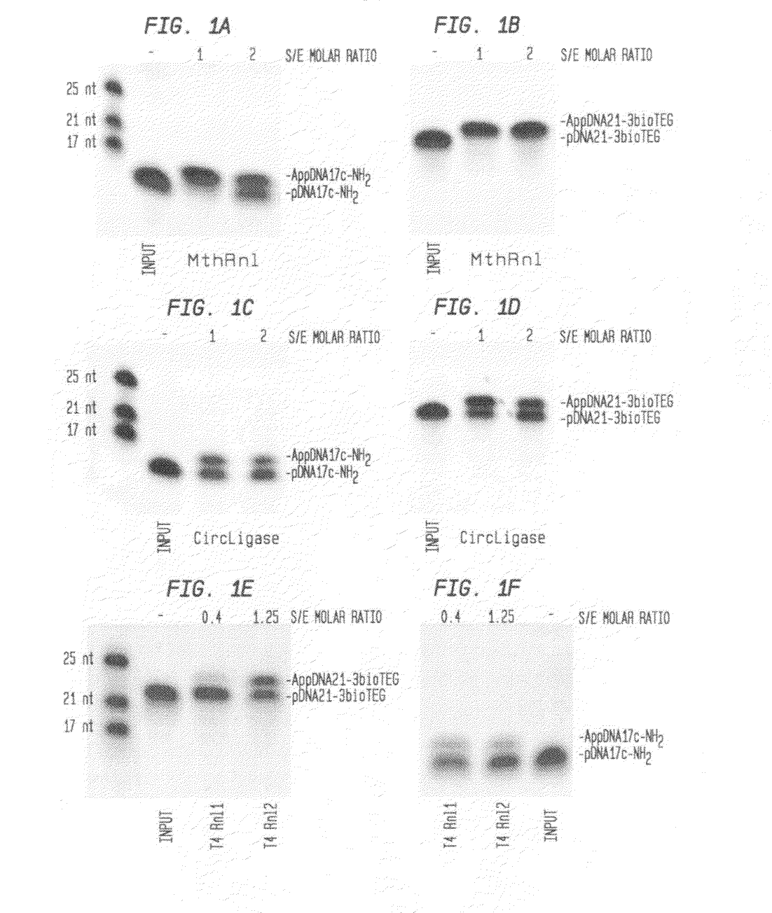

RNA ligases capable of efficiently synthesizing 5′-adenylated oligonucleotides is provided. Initial screening of commercially available RNA ligases (T4 Rnl1, T4 Rnl2, CircLigase™ and MthRnl) showed that all ligases tested were capable for adenylating DNA to some extent although MthRnl produced adenylated product with the highest yield (FIGS. 1A-F).

Reactions were performed using conditions recommended by the manufacturer. In general, reactions were carried out with an equimolar or lesser ratio of substrate to enzyme (S/E). Although MthRnl is known to form a homodimer, the monomer molecular weight was used for calculation of substrate/enzyme molar ratio in the reaction (FIGS. 1A-F).

### Reagents

MthRnl, T4 Rnl1, T4 Rnl2, and T4 Rnl2tr were obtained from NEB, Ipswich, Mass. CircLigase™ was obtained from Epicentre Biotechnologies, Madison, Wis. Oligonucleotides used in this study were synthesized at Integrated DNA Technologies, Coralville, Iowa.

Oligonucleotides used in adenylation experiments:

(SEQ ID NO: 3)
pAGT GAA TTC GAG CTC GGT ACC CGG TGG
ATC CTC TAG AGT CGA CCT GCA GG;
(pDNA50)
(SEQ ID NO: 4)
pTCG TAT GCC GTC TTC TGC TTG-NH₂;
(pDNA21-NH₂)
(SEQ ID NO: 5)
pTCG TAT GCC GTC TTC TGC TTG-bioTEG;
(pDNA21-3bioTEG)
(SEQ ID NO: 6)
pCTA TAG AAA CCC ACG CAA AGC CC-ddC;
(pDNA23-ddC)
(SEQ ID NO: 7)
pCTG TAG GCA CCA TCA AT-NH₂;
(pDNA17c-NH₂)
(SEQ ID NO: 8)
pATG TAG GCA CCA TCA AT-NH₂;
(pDNA17a-NH₂)
(SEQ ID NO: 9)
pTTG TAG GCA CCA TCA AT-NH₂;
(pDNA17t-NH₂)
(SEQ ID NO: 10)
pGTG TAG GCA CCA TCA AT-NH₂.
(pDNA17g-NH₂)

RNA acceptors used in ligation experiments:

(SEQ ID NO: 11)
FAM-CUG AUG AAA CCC ACG CAA AGC CC;
(FAM-RNA23 acceptor)
(SEQ ID NO: 12)
CUA UAC AAC CUA CUA CCU CAA A.
(RNA22 acceptor)

The histidine-tagged MthRnl was expressed in E. coli using a codon-optimized gene and the T7 expression system, and purified according to Torchia et al. (Nucleic Acids Res. 36: 6218-6227 (2008)).

During purification, mass spectrometry was used to assess whether column fractions contained the adenylated or free form of MthRnl. Based on this assay, column fractions were pooled to yield enzyme predominantly in the adenylated or free form.

### Reaction Conditions

Standard oligonucleotide adenylation reactions were performed in reaction mixtures (10 μl total) containing 50 mM sodium acetate, pH 6.0 buffer, 5 pmol of 3′-blocked, 5′-phosphorylated oligonucleotide, 100 μM ATP, 10 mM MgCl₂, 5 mM DTT, 0.1 mM EDTA, 5 pmol (230 ng) of MthRnl (monomer). Assays were performed at 65° C. for 60 min, followed by inactivation of the enzyme by heating at 85° C. for 5 min. After addition of 5 μl formamide loading buffer, the reaction mixture was separated on a 15% Urea-TBE denaturing polyacrylamide minigels (Invitrogen, now Life Technologies, Carlsbad, Calif.), stained with SYBR® Gold (Invitrogen, now Life Technologies, Carlsbad, Calif.) and visualized using an AlphaImager HP (Alpha Innotech, now Cell Biosciences, Santa Clara, Calif.).

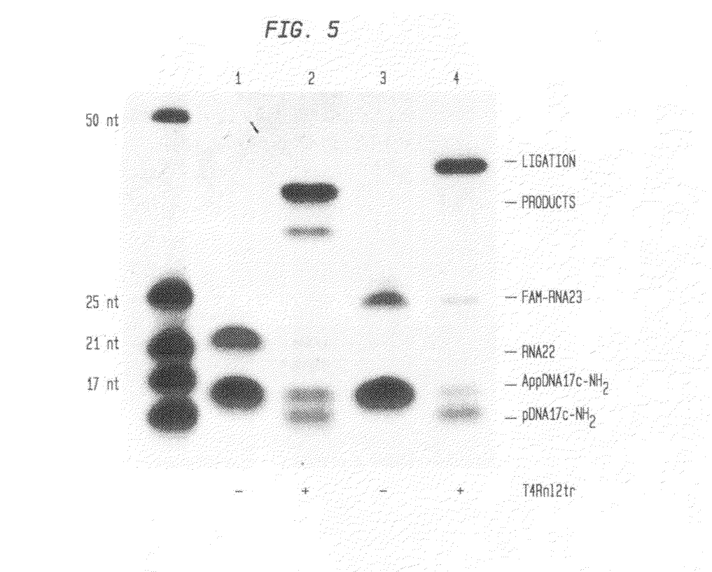

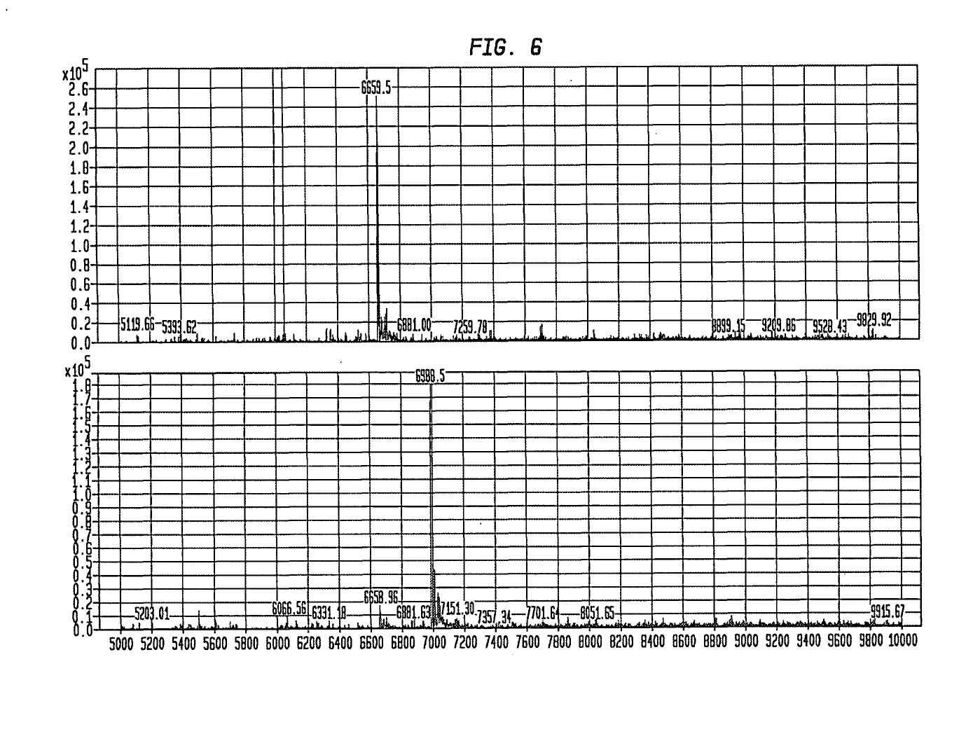

The DNA circularization assay was performed using an excess of pre-adenylated MthRnl (25 pmol monomer), and 5 pmol of 3′-OH, 5′-phosphorylated oligonucleotide, and variable concentrations of ATP. The circularity of ligated DNAs was identified via resistance to Exonuclease I (NEB, Ipswich, Mass.) digestion. The presence of adenylated DNA was confirmed by ESI-MS analysis (FIG. 6) and by functional assays described herein (FIG. 5).

For preparative DNA adenylation, 300 μM of DNA substrate and 150-300 μM of enzyme monomer were used in reactions as described above in appropriately scaled reaction volumes at 65° C. for 2 hours, and the extent of reaction assessed on 5 pmol DNA aliquots by gel electrophoresis. The remainder of the reactions was heat-inactivated at 85° C. for 5 min, treated with Proteinase K (NEB, Ipswich, Mass.), extracted with phenol-chloroform-isopropanol (25:24:1), chloroform-isopropanol (49:1) and ethanol-precipitated.

### Example 2

### Protocol Optimization—Varying Ratios of DNA to MthRnl

MthRnl was incubated in a 10 μl reaction with an oligonucleotide, having a 5′ phosphate and a 3′ NH₂. The reaction contained: 1 μl 10× reaction buffer, 10 μmol of substrate, 1.25-10 μmol of MthRnl (460 ng of ligase=10 pmole of monomer), and 100 μM ATP. The 10× reaction buffer contained 0.50 M NaOAc, pH 6.0, 50 mM DTT, 1 mM EDTA and 0.10 M MgCl₂.

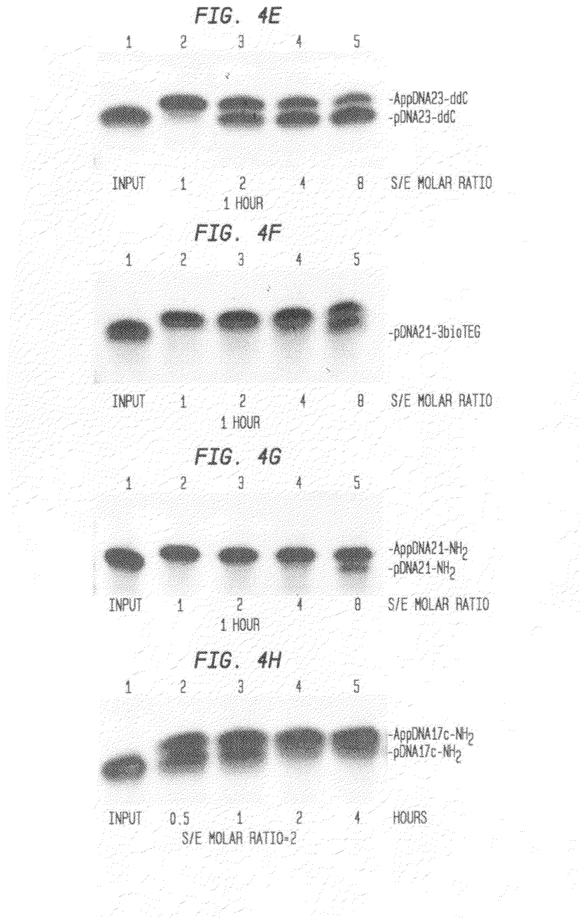

Assays were performed at 65° C. for 60 minutes. The enzyme was inactivated by heating at 85° C. for 5 min. After the addition of 5 μl formamide, reaction products were separated on 15% Urea-TBE denaturing minigels (Invitrogen, now Life Technologies, Carlsbad, Calif.), stained with SYBR® Gold (Invitrogen) and photographed using AlphaImager HP (Alpha Innotech, now Cell Biosciences, Santa Clara, Calif.). The results are shown in FIGS. 1A-1F and 4A-4G. MthRnl was consistently effective at equimolar concentration for the different substrates shown.

### Example 3

### Protocol Optimization—ATP Concentrations

Further optimization of the DNA adenylation reaction was performed for MthRnl, although it is envisaged that the optimized protocol described herein is suited to the entire class of ATP-sensitive RNA ligases described herein. A person of ordinary skill in the art may make minor adjustments with respect to temperature optima according to the thermostability of the RNA ligase.

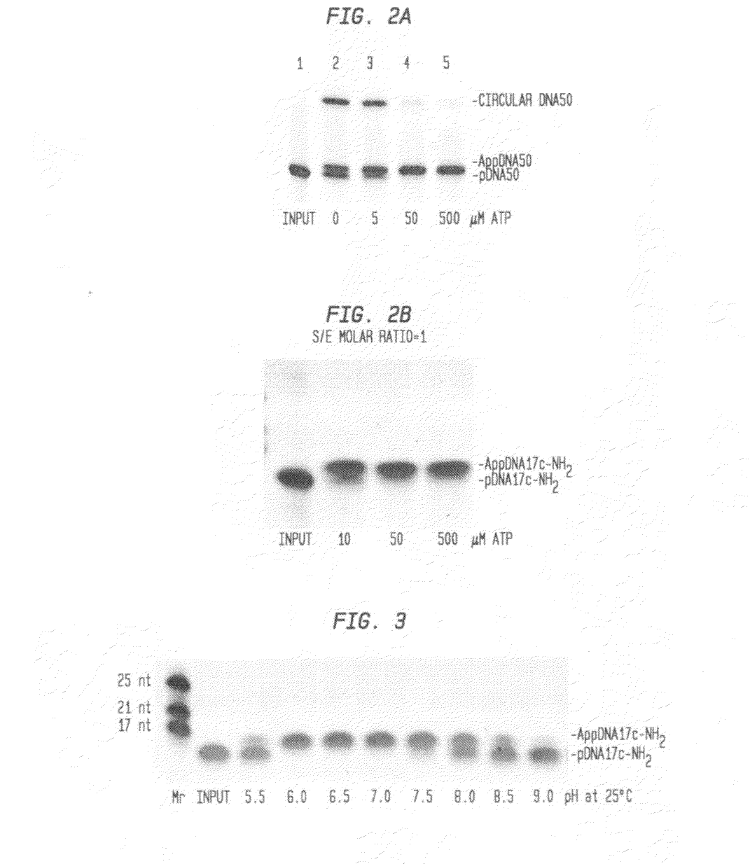

MthRnl was incubated with a 50 nt oligonucleotide with a 5′ phosphate and a 3′ OH, and 17 nt oligonucleotide with 5′ phosphate and 3′ amino block in a 10 μl reaction. The reaction contained: 1 μl 10× reaction buffer, 5 pmol of substrate, and 5 or 10 pmol of MthRnl (460 ng of ligase=10 pmole of monomer). The 10× reaction buffer contained 0.50 M NaOAc, pH 6.0, 50 mM DTT, 1 mM EDTA and 0.10 M MgCl₂. The temperature and time for the assays were the same as Example 1. The reactions contained variable amounts of ATP. The Mth ligase reactions with about 50 μM ATP resulted in enhanced adenylation. An amount of about 500 μM ATP blocked self-ligation of DNA molecules that have a free 3′ OH. The results are shown in FIGS. 2A and 2B.

When the concentration of ATP was increased, two bands were observed, where one product ran one nucleotide slower than substrate on denaturing polyacrylamide gel and corresponded to AppDNA. Synthesis of AppDNA was confirmed by functional analysis (FIG. 5) and mass-spectrometry (FIG. 6).

High ATP concentrations (greater than 5 μM ATP) inhibited DNA circularization with pre-adenylated MthRnl as shown in FIG. 2A. Only trace amounts of circular DNA occurred at 50 μM ATP (FIG. 2A, lane 4). At an ATP concentration of 500 μM, ligation was substantially inhibited (FIG. 2A, lane 5). Adenylation was maximum at ATP concentration of greater than or equal to 10 μM ATP (FIG. 2B). The magnitude of ATP-inhibition of ligation and of AppDNA accumulation, as a function of ATP concentration, was greater for DNA than RNA. DNA substrates with 3′ protected ends showed similar characteristics: AppDNA formation increased with increasing ATP concentration and reached saturation near 50 μM (FIG. 2B).

For MthRnl, ATP is an effective inhibitor of the DNA ligation step. When the concentration of ATP was increased, a product accumulated that ran one nucleotide slower than substrate on denaturing polyacrylamide gel, and presumably is AppDNA (see FIGS. 1A-1F, 2A-B, 3, 4A-4H, and 5 and mass spectrometry (FIG. 6)). The concatamer products which would have resulted from ligation were not observed. DNA circularization was also drastically reduced at high ATP concentrations (FIG. 2A). In summary, increased concentrations of ATP were found to inhibit ligation or circularization of oligonucleotide substrates by ATP-sensitive ligases such as MthRnl.

### Example 4

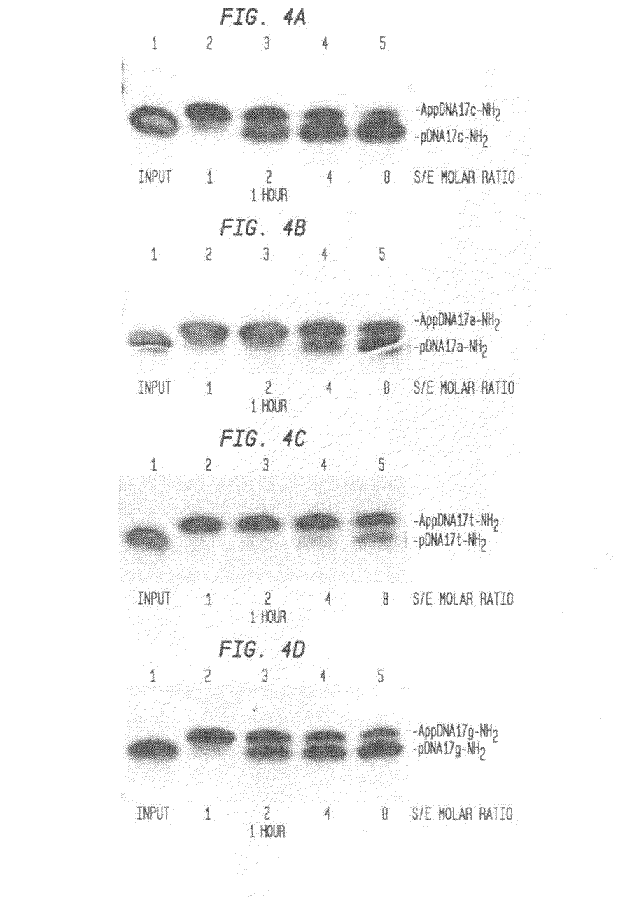

### Protocol Optimization—pH pH optimization is shown in FIG. 3. In the presence of Mg⁺², the pH-optima for DNA adenylation using MthRnl was in the range of 6.5-7.0 in NEBuffer 1 (NEB, Ipswich, Mass.) and pH-adjusted at 25° C. When Mg⁺² was substituted for Mn⁺², pH optima were shifted to 5.5-6.0.

### Example 5

### Functional Assays of MthRnl-Adenylated DNA Oligonucleotide Ligation to RNA

The DNA oligonucleotide adenylated by MthRnl (as described in Example 4) was ligated to RNA by T4 Rnl2tr. 10 μl ligation reactions containing 5 pmol of the RNA acceptor (SEQ ID NO: 11 or SEQ ID NO:12), 7 pmol adenylated pDNA17c-NH₂ in 10 mM Tris-HCl pH 7.5 buffer, 10 mM Mg, 1 mM DTT and 200 U of T4 Rnl2tr were incubated for 2 hours at 25° C. Reactions were stopped by adding 5 μl formamide loading buffer, heat-inactivated at 85° C. for 5 minutes. Ligation reactions were separated on 15% TBE urea containing polyacrylamide gels, and products were visualized by staining with SYBR® Gold (Invitrogen, now Life Technologies, Carlsbad, Calif.) and scanning on a GE Healthcare Life Sciences (Piscataway, N.J.) Typhoon 9400 variable mode imager.

Ligated products were observed in the reaction that contained the MthRnl-adenylated oligonucleotide, RNA and T4 Rnl2tr (FIG. 5, lanes 2 and 4). The ligation experiments demonstrated that oligonucleotides previously reacted with MthRnl were modified such that they were substrates for T4 Rnl2tr.

### Example 6

### ESI-MS Analysis of 5′ Phosphate Oligonucleotide Before and after Reaction with MthRnl Demonstrates 5′ Adenylation

A 5′ phosphorylated 21 nt oligonucleotide, 5′ pTCG TAT GCC GTC TTC TGC TTG-NH₂ (SEQ ID NO:4) 3′ amino block, was reacted with MthRnl under the conditions described in Example 1 using 100 μM ATP. Reacted and unreacted oligonucleotides were analyzed by electrospray ionization mass spectrometry according to the method of Shah and Friedman (Nature Protocols 3(3): 351-6 (2008)). Samples (10 mM oligonucleotides in 50% acetonitrile, 1% triethylamine) were conducted by direct infusion (10 ml/min) into a 6210 ESI-TOF mass spectrometer with an electrospray ionization source (Agilent Technologies, Hollis, N.H.). Data were acquired in negative ion mode, from m/z 500 to 8000 (high mass range enabled). The VCap and Fragmentor values were set to 4000 and 215 V, respectively. The drying gas flow rate was 7 L/min, the gas temperature was 300° C. and the nebulizer was set to 25 psig. The results are shown in the top and bottom panels of FIG. 6. The observed mass increased by 329 Da after reaction with MthRnl in comparison to pDNA substrate, which corresponds to molecular weight of AMP minus H₂O. This is consistent with 5′ adenylation of the oligonucleotide.

### Example 7

### Identification of ATP-Sensitive RNA Ligases for Adenylating Oligonucleotides

Gene databases can be interrogated using part or all of the sequences of the two ATP-sensitive RNA ligases defined by SEQ ID NO: 1 or SEQ ID NO: 2, or using sequences having at least 90% sequence homology with SEQ ID NO: 1 or 2. Candidate enzymes are defined by sequence matches that share at least 90% sequence identity with at least 10% or 20% of SEQ ID NO: 1 or 2. These candidate enzymes can be synthesized and assayed as described herein for MthRnl.

What is claimed:

1. A method for generating an adenylated oligonucleotide preparation, comprising:
(a) providing oligonucleotides having a 5′ phosphate;
(b) reacting the oligonucleotide with an ATP-sensitive ligase in the presence of an effective amount of ATP; and
(c) obtaining a stable reaction product in which greater than 70% of the oligonucleotides are adenylated.

2. A method according to claim 1, wherein the ATP-sensitive ligase is thermostable.

3. A method according to claim 2, wherein the thermostable ligase is an Mth RNA ligase.

4. A method according to claim 1, wherein the effective amount of ATP is sufficient to inhibit circularization or concatamerization and to permit adenylation.

5. A method according to claim 4, wherein the effective amount of ATP is in the range of 5 μM-10 mM ATP.

6. A method according to claim 1, wherein the ATP-sensitive ligase has at least 90% sequence homology with a ligase obtained from at least one of the group consisting of: Methanobacterium thermoautotrophicum; Pyrococcus abyssii; phage KVP40; Deinococcus radiodurans; Autographica California; Rhodothermus marinus; and phage TS2126.

7. The method according to claim 1, wherein the ligase is an Mth RNA ligase or a ligase having at least 90% amino acid sequence similarity to SEQ ID NO: 1 or SEQ ID NO: 2.

8. The method according to claim 1, further comprising: ligating the adenylated oligonucleotide to a polynucleotide by means of a second ligase.

9. The method according to claim 8, wherein the second ligase is a T4 RNA ligase or mutant thereof.

10. The method according to claim 9, wherein the T4 RNA ligase is truncated T4 RNA Ligase 2.

11. The method according to claim 1, wherein adenylation of the oligonucleotide is performed at a temperature in the range of 37° C.-70° C.

12. The method according to claim 1, wherein the oligonucleotide has a blocked 3′ end or a free hydroxyl group at the 3′ end.


SEQUENCE LISTING (14 sequences)

SEQ ID NO: 1 - protein, 395 aa, Bacteriophage TS2126
MSSLAPWRTTSWSPLGSPPSLEDALRLARTTRAFAVRRDGEGRALVTYLYGTPELFSLPGARELRGIVYREEDGTVLSRPFHKFFNFGEPLAPGEEAFKAFRDSMVPLFVAEKVDGYLAQAYLDGGELRFASRHSLNPPLVGALLRKAVDEEAMARLGKLLAAEGGRWTALLEVVDPEAPVMVPYQEPGVYLLALRSIGEGHYLLPGVHFPLPEALRYVRWEPRMDFDPHRFRGEIRDLQGVEGYVVTDGAEFVKFKTGWAFRLARFLMDPEGVFLEAYAEDRLDDLVGALAGREDLLRAVARAQDYLAGLYGEAVGAGDALRRMGLPRKEAWARVQEEAGRWGGFAPAYARAAMAAYEGGEAREAFLVELRKRSARKALEALHLFPRVGGELRG

SEQ ID NO: 2 - protein, 380 aa, Methanobacterium thermoautotrophicum
MNSMNSDIPFDLIQERTGVPSSRLKVAFARGSLRLLESAGMQALLFKKPLGDLEAGTVIYLGDETEVIRGFPKIRRTLLLSPTIQEHFRDRVAVEEKMNGYNVRIACLSSGETVALTRGGHVCPFTTRKAQELLDLSEFFREHPDLVICGEMIGRDNPYVSQDYPEVGPLGFRVFDLREKNTNRPLPVEERRALLDSYGLPNVRLFGVYPIEEAASEVADIIRALGMAGREGVVMKDPSMEVPPLKYSSQAHARELAYAFSYPFDFGRPFFFSRVIREGFQAYELDESDDETRERARRLGEAIIYPMLERIKSISAGEAAYEDTVIDVEDREAAEEFIRHLVRLGVSATLADYRDGRATIRRFYQSTTDRINNYLKGGLY

SEQ ID NO: 3 - DNA, 50 bp, artificial
  oligonucleotide
agtgaattcgagctcggtacccggtggatcctctagagtcgacctgcagg

SEQ ID NO: 4 - DNA, 21 bp, artificial
  oligonucleotide
tcgtatgccgtcttctgcttg

SEQ ID NO: 5 - DNA, 21 bp, artificial
  oligonucleotide
tcgtatgccgtcttctgcttg

SEQ ID NO: 6 - DNA, 23 bp, artificial
  oligonucleotide
ctatagaaacccacgcaaagccc

SEQ ID NO: 7 - DNA, 17 bp, artificial
  oligonucleotide
ctgtaggcaccatcaat

SEQ ID NO: 8 - DNA, 17 bp, artificial
  oligonucleotide
atgtaggcaccatcaat

SEQ ID NO: 9 - DNA, 17 bp, artificial
  oligonucleotide
ttgtaggcaccatcaat

SEQ ID NO: 10 - DNA, 17 bp, artificial
  oligonucleotide
gtgtaggcaccatcaat

SEQ ID NO: 11 - RNA, 23 nt, artificial
  acceptor
cugaugaaacccacgcaaagccc

SEQ ID NO: 12 - RNA, 22 nt, artificial
  acceptor
cuauacaaccuacuaccucaaa

SEQ ID NO: 13 - RNA, 26 nt, artificial
  oligonucleotide
guucagaguucuacaguccgacgauc

SEQ ID NO: 14 - DNA, 22 bp, artificial
  oligonucleotide
tcgtatgccgtcttctgcttgt